(19) United States
(12) Patent Application Publication
(10) Pub. No.: US 2009/0112326 A1
(43) Pub. Date: Apr. 30, 2009

(54) IN SITU ADJUSTABLE DYNAMIC INTERVERTEBRAL IMPLANT

(75) Inventors: Jean-Charles Lehuec, Bordeaux (FR); Erik O. Martz, Savage, MN (US)

Correspondence Address: FAEGRE & BENSON LLP;PATENT DOCKETING, 2200 WELLS FARGO CENTER, 90 SOUTH SEVENTH STREET, MINNEAPOLIS, MN, 55402-3901, US

(73) Assignee: Disc Dynamics, Inc., Eden Prairie, MN (US)

(21) Appl. No.: 12/014,560

(22) Filed: Jan. 15, 2008

Related U.S. Application Data
(60) Provisional application No. 60/982,359, filed on Oct. 24, 2007.

(51) Int. Cl. A61F 2/44

(52) U.S. Cl. 623/17.16; 623/17.11; 623/17.13

(57) ABSTRACT

A system for forming a spinal prosthesis in situ within an intervertebral space located between first and second adjacent vertebrae includes at least one mold having at least one internal compartment adapted to receive at least one flowable biomaterial. The system also includes a retaining member adapted to secure the mold between the first and second vertebrae, the retaining member including first and second portions adapted to be engaged with first and second surfaces of the first and second vertebrae, respectively. The retaining member also includes an intermediate body operatively coupling the first portion to the second portion, the intermediate body adapted to be positioned in or adjacent to the intervertebral space. A biomaterial delivery apparatus is in fluid communication with the mold at a pressure sufficient for the mold to engage with the retaining member. The spinal prosthesis selectively position the first vertebrae relative to the second vertebrae.

The present application claims the benefit of U.S. Provisional Application Ser. No. 60/982,359 entitled IN SITU ADJUSTABLE DYNAMIC INTERVERTEBRAL IMPLANT, filed on Oct. 24, 2007, which is hereby incorporated by reference.

### TECHNICAL FIELD

The present invention relates to dynamic spinal implants, as well as methods for making in situ adjustments during implantation. More specifically, the invention relates to a combination retaining member and inflatable device that permits in situ adjustment of the spinal implant.

### BACKGROUND OF THE INVENTION

In lateral profile and in a natural state, the vertebral column extends through several curves corresponding generally to the cervical, thoracic, lumbar, and pelvic regions. The cervical curve generally begins at the apex of the odontoid process, and ends at the second thoracic vertebra. The cervical curve can be described as a lordotic curve, being naturally convex in the anterior direction. The thoracic curve generally begins at the second thoracic vertebra and ends at the twelfth thoracic vertebra. The thoracic curve can be described as a kyphotic curve, being naturally concave in the anterior direction. The lumbar curve generally begins at the twelfth thoracic vertebra and ends at the sacrovertebral articulation. The lumbar curve can also be described as a lordotic curve, being naturally convex in the anterior direction. The pelvic curve generally begins at the sacrovertebral articulation, and ends at the point of the coccyx. The pelvic curve can also be described as a kyhpotic curve, being naturally convex in the anterior and downward direction.

The adjacent vertebrae of the spinal column are separated by intervertebral discs, which help maintain the curvature of the spine, provide structural support, and distribute forces exerted on the spinal column. An intervertebral disc generally consists of three major components: opposing vertebral endplates, a nucleus pulposus between the endplates, and an annulus fibrosus extending about the nucleus pulposus and between the endplates.

The central portion, the nucleus pulpous or nucleus is relatively soft and gelatinous; being composed of about 70 to 90% water. The nucleus pulpous has a high proteoglycan content and contains a significant amount of Type II collagen and chondrocytes. Surrounding the nucleus is the annulus fibrosus, which has a more rigid consistency and contains an organized fibrous network of approximately 40% Type I collagen, 60% Type II collagen, and fibroblasts. The annular portion serves to provide peripheral mechanical support to the disc, afford torsional resistance, and contain the softer nucleus while resisting its hydrostatic pressure.

Intervertebral discs, however, are susceptible to a number of injuries that may require partial or total disc replacement. Disc herniation occurs when the nucleus begins to extrude through an opening in the annulus, often to the extent that the herniated material impinges on nerve roots in the spine or spinal cord. The posterior and posterio-lateral portions of the annulus are most susceptible to attenuation or herniation, and therefore, are more vulnerable to hydrostatic pressures exerted by vertical compressive forces on the intervertebral disc. Various injuries and deterioration of the intervertebral disc and annulus fibrosus are discussed by Osti et al., Annular Tears and Disc Degeneration in the Lumbar Spine, J. Bone and Joint Surgery, 74-B(5), (1982) pp. 678-682; Osti et al., Annulus Tears and Intervertebral Disc Degeneration, Spine, 15(8) (1990) pp. 762-767; Kamblin et al., Development of Degenerative Spondylosis of the Lumbar Spine after Partial Discectomy, Spine, 20(5) (1995) pp. 599-607.

One treatment for intervertebral disc injury is directed toward fusion of the adjacent vertebrate, e.g., using a cage in the manner provided by Sulzer. Sulzer's BAK® Interbody Fusion System involves the use of hollow, threaded cylinders that are implanted between two or more vertebrae. The implants are packed with bone graft to facilitate the growth of vertebral bone. Fusion is achieved when adjoining vertebrae grow together through and around the implants, resulting in stabilization, such as for example U.S. Pat. No. 5,425,772(Brantigan) and U.S. Pat. No. 4,834,757(Brantigan).

U.S. Patent Publication No. 2005/0125063(Matge et al.) discloses a dynamic intervertebral implant for a total disc replacement. The metal structure is implanted in place of the entire intervertebral disc. Anchors are typically provided to prevent expulsion of the device. One embodiment of this device is an improvement over traditional fusion devices in that the implant deforms to permit slight movement of the adjacent vertebrae.

PCT Publication No. WO 01/62190 discloses another dynamic intervertebral implant for a total disc replacement. A metal anchor structure is used to secure a preformed viscoelastic core to the adjacent vertebrae.

U.S. Pat. No. 5,645,599 discloses a U-shaped anchor structure used to secure a preformed elastic member between adjacent spinous processes.

### BRIEF SUMMARY OF THE INVENTION

Some aspects of the invention relate to spinal prosthetic systems, methods, and devices. For example, one aspect of the invention relates to a system for forming a spinal prosthesis in situ within an intervertebral space located between first and second adjacent vertebrae. In some embodiments, the system includes at least one mold having at least one internal compartment adapted to receive at least one flowable biomaterial. The system also includes a retaining member adapted to secure the mold between the first and second vertebrae. The retaining member includes a first portion adapted to be engaged with a first surface of the first vertebra and a second portion adapted to be engaged with a second surface of the second vertebra. The retaining member also includes an intermediate body operatively coupling the first portion to the second portion, the intermediate body adapted to be positioned in or adjacent to the intervertebral space. A biomaterial delivery apparatus is in fluid communication with the mold at a pressure sufficient for the mold to engage with the retaining member. The spinal prosthesis selectively position the first vertebrae relative to the second vertebrae.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE SEVERAL VIEWS OF THE DRAWING(S)

FIG. 1 is a perspective exploded view of a system for in situ spinal prosthetic formation within an intervertebral space, according to some embodiments of the invention.

FIG. 2 is a cross-sectional view of a human body taken through the intervertebral space, according to some embodiments of the invention.

FIG. 3 shows a retaining member of the spinal prosthetic of FIG. 1 from a side view, according to some embodiments of the invention.

FIG. 4 is a cross-sectional view of the retaining member along line 4-4 of FIG. 3, according to some embodiments of the invention.

FIG. 5 shows the retaining member of FIG. 3 from a front view, according to some embodiments of the invention.

FIG. 6 is a schematic fluid circuit diagram of a biomaterial delivery apparatus of the system of FIG. 1, according to some embodiments of the invention.

FIG. 7 is a perspective view of a core member and a portion of the delivery apparatus of FIG. 6, according to some embodiments of the invention.

FIGS. 8-11 are cross-sectional, side views illustrative of methods of spinal prosthetic implantation and formation, according to some embodiments of the invention.

FIG. 12 shows another system for in situ spinal prosthetic formation within an intervertebral space, according to some embodiments of the invention.

FIG. 13 shows a portion of a spinal prosthetic of the system of FIG. 12, according to some embodiments of the invention.

FIGS. 14A-14C are a cross-sectional, top view of the spinal prosthetic of the system of FIG. 12, according to some embodiments of the invention.

FIGS. 15A-18B are perspective and side views of various retaining members, according to some embodiments of the invention.

FIG. 19 is a perspective view of another spinal prosthetic, according to some embodiments of the invention.

FIG. 20 shows a retaining member of the spinal prosthetic of FIG. 19, according to some embodiments of the invention.

FIG. 21 is a sectional side view of another spinal prosthetic, according to some embodiments of the invention.

FIG. 22 is a sectional side view of another spinal prosthetic, according to some embodiments of the invention.

FIGS. 23a and 23b show optional end features of the spinal prosthetic of FIG. 22, according to some embodiments of the invention.

FIG. 24 shows another configuration of the spinal prosthetic of FIG. 22, according to some embodiments of the invention.

FIGS. 25 and 26 show a sectional side view and a front view, respectively, of another spinal prosthetic, according to some embodiments of the invention.

FIG. 27-35 show other spinal prosthetics from a sectional side view, according to some embodiments of the invention.

FIGS. 36-39 show retaining members of other spinal prosthetics, according to some embodiments of the invention.

FIG. 40-45 show other spinal prosthetics usable with adjacent spinous processes, according to some embodiments of the invention.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION OF THE INVENTION

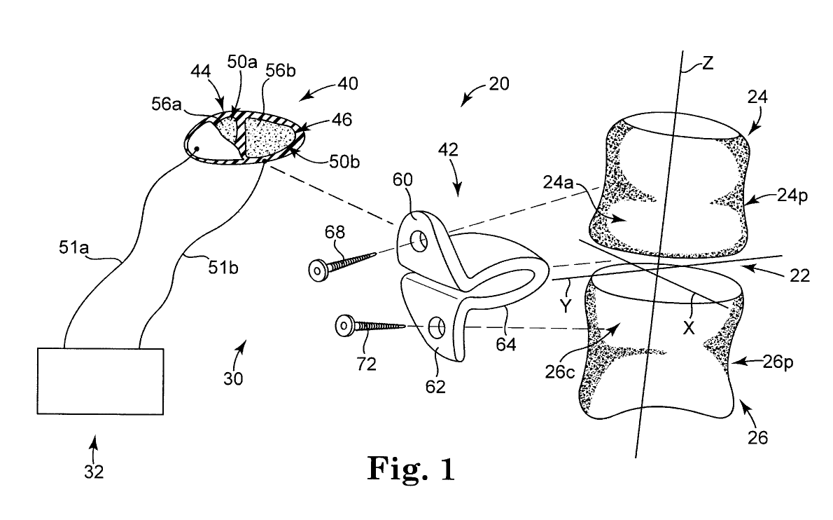

FIG. 1 shows a perspective, unassembled view of a system 20 for in situ spinal prosthetic formation within an intervertebral space 22 defined between a first vertebra 24 and second vertebra 26 according to one embodiment of the present invention. In some embodiments, the first and second vertebrae 24, 26 are cervical vertebrae and prosthetic implantation is performed to help ensure a desired load bearing capability, spacing, and/or lordotic curvature between the first and second vertebra 24, 26. Although the system 20 and associated methods of prosthetic implantation are generally described in association with the cervical region, similar principles are applicable to embodiments addressing other spinal regions, or even other bodily structures, such as knee joints, for example.

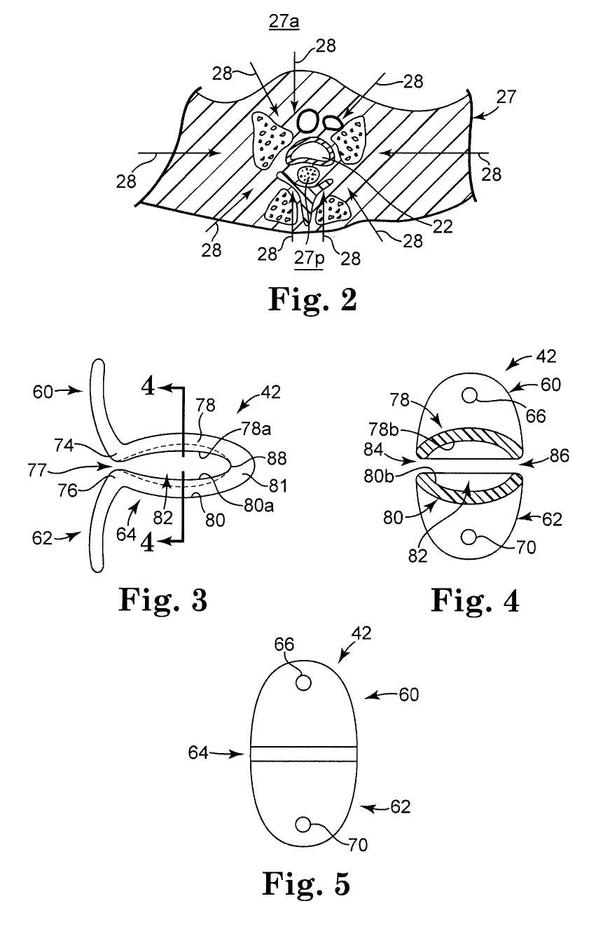

As used herein, the term “anterior” generally refers to an orientation toward the front of the body while “posterior” refers to an orientation toward the back of the body. FIG. 2 is a cross-sectional view of a human body 27 taken through the intervertebral space 22. As understood by those of skill in the art, the body 27 has an anterior side 27a, or front side 27a, and a posterior side 27p, or back side 27p. There are a variety of access paths 28 to the intervertebral space 22 for prosthetic implantation that are known to those of skill in the art.

With combined reference to FIGS. 1 and 2, the first and second vertebrae 24, 26, and in particular the endplates of each of the first and second vertebrae 24, 26, define the upper and lower boundaries of the intervertebral space 22. The first and second vertebrae 24, 26 each define anterior faces 24a, 26a, respectively, and posterior faces 24p, 26p, respectively. The intervertebral space 22 has a posterior-anterior axis, or Y-axis, a latero-lateral axis, or X-axis, and a rostro-caudal axis, or Z-axis, as well as rotation around each of the X axis (pitch), Y axis (roll) and Z axis (yaw).

As shown in FIG. 1, the system 20 includes a spinal prosthetic 30 (shown in an unassembled state) and a biomaterial delivery apparatus 32 in fluid communication with the prosthetic 30. The prosthetic 30 includes a core member 40 and a retaining member 42. In general terms, the retaining member 42 is adapted to be secured to the first and second vertebrae 24, 26 and to assist with retaining the prosthetic 30 in the intervertebral space 22. In some embodiments, the prosthetic 30 acts to carry all or a portion of spinal loads placed on the intervertebral space 22. In other embodiments, the retaining member 42 is adapted to assist with this load-bearing function, also carrying a portion of the spinal loads.

The core member 40 includes a mold 44 (shown partially cut away in FIG. 1) and a biomaterial system 46 housed within the mold 44. In some embodiments, the mold 44 is generally balloon-like in nature that can transition between a collapsed state to an expanded state upon injection of the biomaterial system 46 into the mold 44. The mold 44 is formed of a variety of materials, including biocompatible polymeric materials that are compliant or non-compliant, and other materials. In some embodiments where the mold 44 is non-compliant, the mold material is characterized as substantially rigid and unable to be expanded beyond a predefined geometry. Suitable molds, mold materials and biomaterials are described in U.S. Pat. No. 5,556,429 (Felt); U.S. Pat. No. 6,306,177 (Felt, et al.); U.S. Pat. No. 6,248,131 (Felt, et al.); U.S. Pat. No. 5,795,353 (Felt); U.S. Pat. No. 6,079,868 (Rydell); U.S. Pat. No. 6,443,988 (Felt, et al.); U.S. Pat. No. 6,140,452 (Felt, et al.); U.S. Pat. No. 5,888,220 (Felt, et al.); U.S. Pat. No. 6,224,630 (Bao, et al.); U.S. Pat. No. 7,001,431 (Bao et al.); and U.S. Pat. No. 7,077,865 (Bao et al.); U.S. Patent Publication No. 2006/0253199 entitled Lordosis Creating Nucleus Replacement Method and Apparatus; and U.S. application Ser. No. 11/420,055, filed May 24, 2006, entitled Mold Assembly for Intervertebral Prosthesis, all of which are hereby incorporated by reference.

In the illustrated embodiment, the mold 44 includes a first internal compartment 50a and a second internal compartment 50b(collectively, “internal compartments 50”). In some embodiments, the mold 44 includes an internal partition 52, also described as a septum, dividing the first and second compartments 50a, 50b. Although multiple compartments are shown, in some embodiments, the mold 44 includes a single, unitary compartment. Multiple molds 44 can be used in place of multi-compartment molds. As used herein, reference to multiple internal compartment means a single mold with multiple compartments and/or multiple discrete molds. Additional molds 44 suited for use with the spinal prosthetic 30 are disclosed in U.S. Patent Publication No. 2006/0253198, entitled Multi-Lumen Mold For Intervertebral Prosthesis And Method Of Using Same, previously incorporated by reference.

The biomaterial system 46 optionally includes a radio-opaque filler or otherwise has radio-opaque properties and is adapted to be delivered in a fluid form, where the biomaterial system 46 is initially flowable into the mold 44 in situ and can then be cured to achieve desired properties. In other embodiments, the bio material system 46 is non-curable. The biomaterial system 46 includes one or more biomaterials 56, such as a first biomaterial 56a disposed in the first compartment 50a and a second biomaterial 56b disposed in the second compartment 50b, although systems including fewer or greater biomaterials are also contemplated. In the illustrated embodiment, biomaterial delivery apparatus 32 is connected to the first and second compartments 50a, 50b by separate lumens 51a, 51b. As will be discussed below, the ability to control delivery of the biomaterial 46 to each compartment 50a, 50b permits in situ adjustment of the spinal prosthetic 30.

In some embodiments, the first and second biomaterials 56a, 56b are similar. In other embodiments, the first and second biomaterials 56a, 56b are characterized by substantially different mechanical, chemical, or other properties. For example, the first biomaterial 56a is substantially more rigid than the second biomaterial 56b in some embodiments. In a related embodiment, the first biomaterial 56a is characterized by a substantially higher spring constant (k) than the second biomaterial 56b. In another related embodiment, the first biomaterial 56a is characterized by a substantially higher modulus of elasticity (E) than the second biomaterial 56b.

As will be described in greater detail, the configuration of the first and second biomaterials 56a, 56b can be differentiated to assist with load balancing in the intervertebral space 22. In some embodiments, load balancing techniques help provide relatively more posterior or anterior support in the intervertebral space 22, which can also help reduce the potential for migration or expulsion of the core member 40 from the intervertebral space 22.

Although some embodiments include an inflatable core member 40 having a mold 44 and a biomaterial system 46, other embodiments include a core member formed of a deflated or dehydrated implant adapted to expand within the retaining member 42 following implantation. In some embodiments, the core member is pre-assembled, or pre-formed as a solid piece that is subsequently assembled in the retaining member 42.

In general, the retaining member 42 is adapted to secure the core member 40 between the first and second vertebrae 24, 26. In particular, the retaining member 42 includes a first flange 60 that is adapted to be secured to the first vertebra 24, a second flange 62 opposite the first flange 60 that is adapted to be secured to the second vertebra 26, and an intermediate body 64 that is adapted to be positioned at least partially in the intervertebral space 22.

FIG. 3 shows the retaining member 42 from a side view. FIG. 4 shows a cross-section of the retaining member 42 along line 4-4 of FIG. 3. FIG. 5 shows the retaining member 42 from a front view. With combined reference to FIGS. 3-5, the first and second flanges 60, 62 are shown positioned opposite one another, each extending in opposite directions from the intermediate body 64. Each of the first and second flanges 60, 62 is U-shaped in front profile and is substantially arcuate in side profile or otherwise adapted to fit against, or track the profile of the vertebrae 24, 26 (FIG. 1). For example, the first and second flanges 60, 62 are substantially convex in an anterior direction when viewed from the side, such that each of the flanges 60, 62 has a shape that substantially conforms to the anterior faces 24a, 26a of the first and second vertebrae 24, 26, respectively.

The first flange 60 has a hole 66 for receiving a bone screw 68 (FIG. 1) while the second flange 62 has a hole 70 for receiving a bone screw 72 (FIG. 1) or other fastener. As will be described in greater detail, the bone screws 68, 72 are inserted through the holes 66, 70 and screwed into the first and second vertebrae 24, 26 (FIG. 1) to secure the retaining member 42 to the vertebrae 24, 26 and relative to the intervertebral space 22 (FIG. 1).

As shown in FIG. 3, the intermediate body 64 has a recurved shape, the intermediate body 64 extending through an arcuate path and being substantially C-shaped in side profile. The intermediate body 64 extends from a first end 74 to a second end 76 and includes an upper portion 78 and a lower portion 80. The first end 74 is connected to the first flange 60 while the second end 76 is connected to the second flange 62. The first and second ends 74, 76 are separated by a gap 77.

The upper and lower portions 78, 80 are each cup-shaped, having inwardly concave shapes 78a, 80a from a side profile (FIG. 3) and inwardly concave shapes 78b, 80b in front cross-section (FIG. 4). In some embodiments, the upper portion 78 is shaped to substantially conform or otherwise track with a shape, e.g., concave profile, of the endplate of the first vertebra 24 while the lower portion 80 is shaped to substantially conform or otherwise track with a shape, e.g., concave profile, of the endplate of the second vertebra 26.

The upper and lower portions 78, 80 are connected at a bend 81. The upper and lower portions 78, 80 also combine to define an interior 82, or recess 82, adapted to receive and retain the core member 40. As shown in FIG. 3, the interior 82 has a tear-drop shape when viewed from a side profile. The interior 82 has an open first side 84, an open second side 86 opposite the first side 84, a front at the gap 77, and a closed back 88. In an embodiment where the retaining member 42 is implanted through a lateral opening, the flanges 60, 62 are typically located laterally, but the bend 81 is preferably located at the anterior or posterior side of the disc space.

In some embodiments, the intermediate body 64 incorporates some flex, or a spring action. In particular, the intermediate body 64 is characterized by a spring action between the upper and lower portions 78, 80, such that the first and second ends 74, 76 can be flexed, or moved toward and away from one another, during spinal loading. The geometry and material of the intermediate body 64, for example, at the bend 81, is selected to control elastic compression and distension of the upper and lower portions 78, 80 toward and away from one another. For example, the intermediate body 64 is made of a material having suitable spring-like qualities, including metals such as stainless steel or suitable polymeric materials.

In other embodiments, the intermediate body 64 does not have sufficient rigidity to support the adjacent vertebrae. For example, the intermediate body 64 may include a geometry and/or a material (e.g., sufficiently flexible) that does not facilitate elastic deflection of the intermediate body 64 during use. For example, the intermediate body 64 is optionally formed of a woven fabric or thin sheet material that does not otherwise exhibit a spring action in use.

One or both of the concavities 78a, 78b, 80a 80b of the upper and lower portions 78, 80 help retain the core member 40 (FIG. 1) within the interior 82 following implantation. For example, the inwardly concave shapes 78a, 80a provides a grasping action to reduce the risk of migration or expulsion through the gap 77 by gripping or otherwise engaging the front of the core member 40. The closed back 88 helps prevent migration or expulsion opposite the gap 77. The concave shapes 78b, 80b help prevent migration or expulsion through the open sides 84, 86 by gripping or otherwise engaging the sides of the core member 40. The spring action also helps prevent migration or expulsion by controlling or limiting the relative angle between vertebrae as will be described in greater detail. Additional or alternate features, adhesives, surface roughening, connectors, or others, can also be employed to reduce the possibility of migration or expulsion of the core member 40 from the interior 82, in turn reducing the risk of core migration or expulsion from the intervertebral space 22 (FIG. 1). For cervical applications, the bend 81 is preferably positioned anteriorly. For lumbar applications, the bend 81 is preferably positioned posteriorly. Any of the access paths 28 can be used with the present method and apparatus.

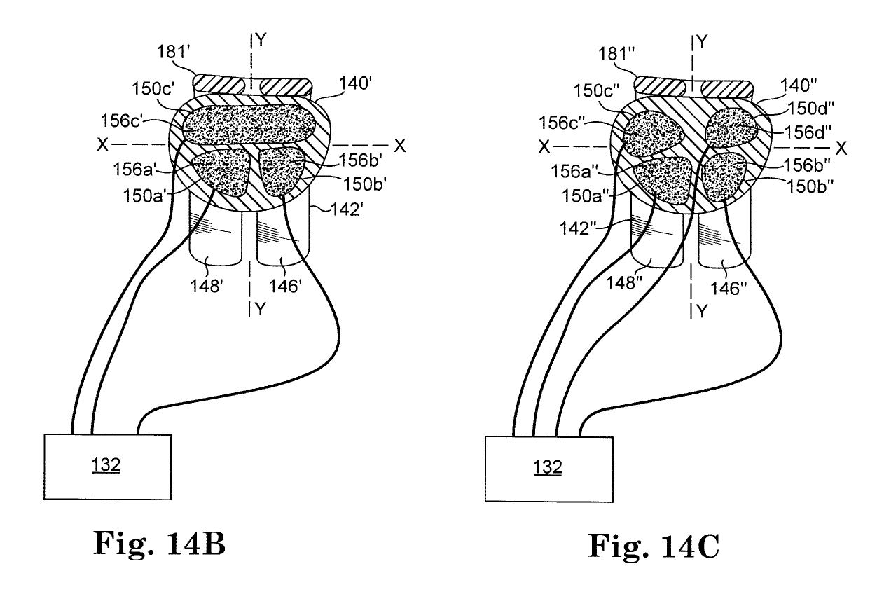

The concavities 78a, 78b, 80a 80b also assist in adjusting the adjacent vertebrae 24, 26 (see FIG. 1) in all six degrees of freedom (X, Y, Z, pitch, roll, yaw). In particular, in embodiments with multiple molds and/or molds with multiple compartments, such as for example illustrated in FIGS. 10 and 14A-14B, the shape of the concavities 78a, 78b, 80a 80b permit more control over the loads transferred between the core member 40 and the adjacent vertebrae 24, 26.

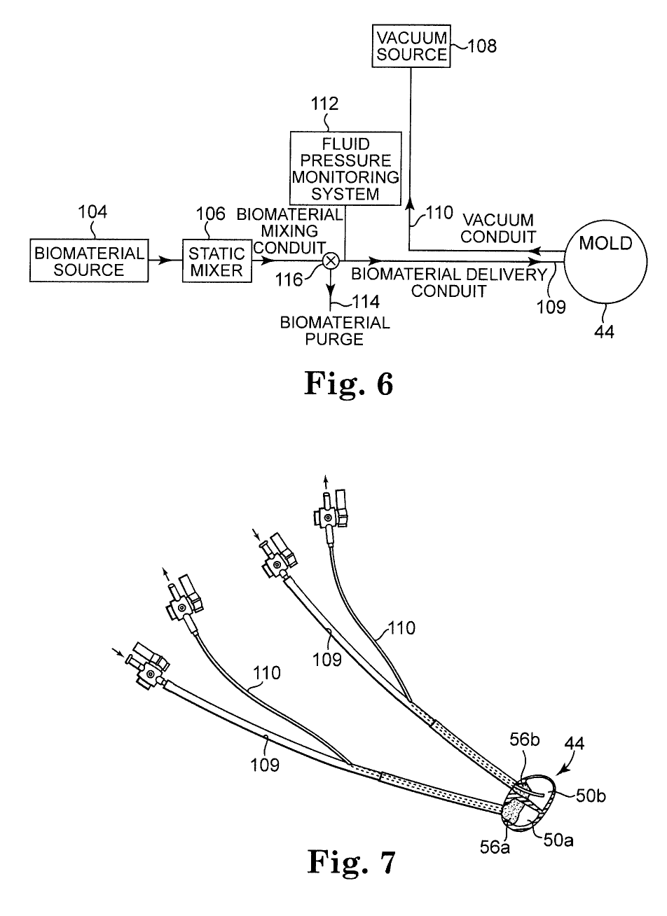

FIG. 6 is a fluid circuit diagram of the biomaterial delivery apparatus 32. In general terms, the apparatus 32 is used for forming and injecting a plurality of biomaterials into the mold 44. The apparatus 32 can include separate components designated for forming and injecting one biomaterial, or can include one or more common components used in forming and injecting multiple biomaterials. In particular, the apparatus 32 is attached to the mold 44 and includes one or more biomaterial sources 104 and one or more static mixers 106 for use in mixing a plurality of components making up the first and second biomaterials 56a, 56b (FIG. 1).

The circuit also includes one or more vacuum sources 108 and associated vacuum conduits 110 and one or more purge paths 114. Control valve(s) 116 are used to access the various conduits in the course of controlling and/or monitoring the pressure and the flow of the first and second biomaterials 56a, 56b through one or more delivery conduits 109 to the mold 44. The circuit also includes one or more endpoint monitors 112 adapted to provide an indication of an endpoint for biomaterial delivery.

In some embodiments, the endpoint monitor 112 is operably attached to the delivery conduit(s) 109 and is a pressure monitor for use in measuring fluid pressure within the conduit(s) 109 and/or the mold 44. In general terms, the endpoint monitor 112 is adapted to provide an indication of when the mold 44 has been expanded a desired amount, or is in a sufficiently expanded state. Suitable pressure monitors include any device or system adapted to measure or indicate fluid pressure within a surgical fluid system and adapted for attachment to a surgical system cannula. Examples of suitable pressure monitors include, but are not limited to, those involving a suitable combination of pressure gauge, electronic pressure transducer and/or force transducer components.

Examples of suitable fluid delivery apparatuses and their workings are described in U.S. Pat. No. 7,001,431, “Intervertebral Disc Prosthesis,” and U.S. Patent Publication No. 2005/0209602, entitled “Multi-Stage Biomaterial Injection System for Spinal Implants, both of which are incorporated by reference.

FIG. 7 is a perspective view showing first and second vacuum conduits 110a, 110b and first and second delivery conduits 109a, 109b. The conduits 109a, 109b, 110a, 110b, are used for delivering the first biomaterial 56a to the first compartment 50a and the second biomaterial 56b to the second compartment 50b of the mold 44 using complementary injection/vacuum techniques similar to those described in previously incorporated U.S. Pat. No. 7,001,431.

In some embodiments, implanting and forming the prosthetic 30 in vivo includes accessing the intervertebral space 22 via one or more access paths 28 and removing at least a portion of the disc annulus (not shown) and at least a portion of the disc nucleus (not shown) according to any of a variety of techniques known to those of skill in the art. It will be understood that certain combinations of the access paths 28 are preferred depending on a number of factors, such as the nature of the procedure, the patient's condition, and others.

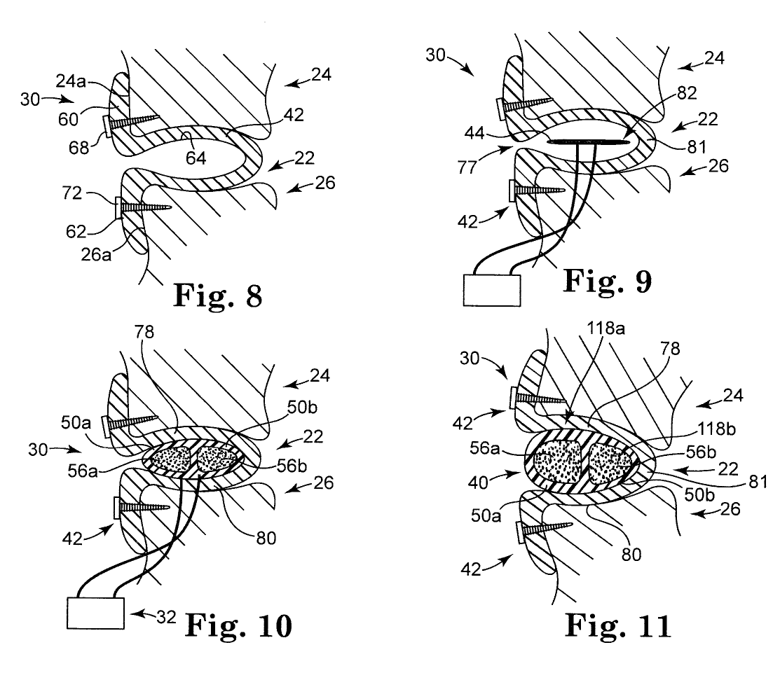

FIGS. 8-11 are cross-sectional, side views of the intervertebral space 22 between the first and second vertebrae 24, 26 that are referenced in describing embodiment methods of prosthetic implantation and formation. As show in FIG. 8, the retaining member 42 is guided to the first and second vertebrae 24, 26 and the intermediate body 64 is inserted into the intervertebral space 22. The first and second flanges 60, 62 are secured to the anterior faces 24a, 26a of the first and second vertebrae 24, 26, respectively, using the bone screws 68, 72 or other suitable fastening means, including adhesives, clamps, and others.

In some embodiments, distraction of the first and second vertebrae 24, 26, for example, using known techniques and devices, is performed to facilitate insertion of the intermediate body 62. The vertebrae 24, 26 are optionally prepared to promote in-growth or otherwise improve fixation of the retaining member 42 to the vertebrae 24, 26. For example, the endplates and/or other portions of the vertebrae 24, 26 are optionally milled or roughened prior to or during implantation of the retaining member 42. Additionally or alternatively, growth or friction promoting coatings or other surface treatments are optionally applied. Although FIG. 8 shows the retaining member 42 in the intervertebral space 22 without the mold 44, in some embodiments, the mold 44 is pre-assembled into the retaining member 42 prior to delivering the retaining member 42 to the first and second vertebrae 24, 26.

As shown in FIG. 9, the mold 44 is received within the interior 82 defined by the recurved shape of the retaining member 44. In some embodiments, the mold 44 is disposed in the interior 82 of the retaining member 42 with the first compartment 50a (FIG. 10) oriented toward the gap 77 and the second compartment 50b (FIG. 10) oriented toward the bend 81. The mold 44 is optionally disposed in the interior 82 through the gap 77 or one of the open sides 84, 86 (FIG. 4).

In some embodiments, prior to installation of the mold 44, an imaging, or trial mold (not shown) is inserted into the retaining member 42 and inflated with contrast material (not shown) to allow fluoroscopic viewing. In particular, the trial mold is optionally inflated to desired fill parameters, for example, a desired fill pressure, prior to installation of the mold 44 in the retaining member 42.

As shown in FIG. 10, the biomaterial delivery apparatus 32 is then used to inject the first and second biomaterials 56a, 56b into the first and second compartments 50a, 50b of the mold 44, inflating the mold against the upper and lower portions 78, 80 of the retaining member 42. In other embodiments, the delivery apparatus 32 or other apparatus is used to inject other fluids, air, non-curing biomaterials, and/or contrast materials, for example. In some embodiments, the injection is performed in vivo after the retaining member 42 and mold 44 have been implanted. The mold can be inflated with biomaterial while the spine of the patient is in a natural lordotic or kyhpotic position, depending on the region of the spine being repaired. The biomaterial can also be injected while the spine of the patient is under a natural load, for example with the patient in a partially or completely upright position.

As shown in FIG. 11, in some embodiments, the first and second biomaterials 56a, 56b are injected with a sufficient volume and/or pressure to cause distraction, or expansion of the upper and lower portions 78, 80, and in particular, the first and second ends 74, 76 apart from one another. As the upper and lower portions 78, 80 move apart, there is an increase in the size of the interior 82 corresponding to the volume of the core member 40.

The biomaterial injection pressure can be used to control a desired amount of distraction pressure in the intervertebral disc space 22, and thus an amount of separation of the first and second vertebrae 24, 26, as well as the curvature or angular offset between the vertebrae 24, 26. In particular, the injection volume and/or pressure of the biomaterials 56a, 56b can be selected to provide a desired amount of angular offset between the upper and lower portions 78, 80 of the retaining member 42.

As the upper and lower portions 78, 80 expand the retaining member 42 presses against the first and second vertebrae 24, 26. This physical engagement engenders a desired spacing between the vertebrae 24, 26. In some embodiments, the sizes of the compartments 50a, 50b, the injection pressures, and/or the relative injection volumes of the first and second biomaterials 56a, 56b are selected to engender a desired degree of angular offset or pitch between the first and second vertebrae 24, 26 around the X-axis, which can otherwise be described as a degree of lordotic curvature or kyphotic curvature between the first and second vertebrae 24, 26. For example, if the intervertebral spacing is selected to be greater anteriorly than posteriorly (e.g., by filling the first compartment 50a with a greater volume of biomaterial than the second compartment 50b) the vertebrae 24, 26 will exhibit a greater degree of lordotic curvature. In other embodiments, spacing is varied to cause a greater degree of kyphotic curvature or an abnormal lateral curvature of the spine in a frontal or mediolateral plane.

The geometry of the retaining member 42 can also be selected according to a desired degree of lordotic or kyphotic curvature. For example, the retaining member 42 can be pre-formed with the upper and lower portions 78, 80 defining a pre-selected angle corresponding to a desired degree of lordotic or kyphotic curvature between the vertebrae 24, 26.

Injection of the first and second biomaterials 56a, 56b continues as desired with curing of the first and second biomaterials 56a, 56b proceeding according to a desired cure rate to form the cured, final core member 40 within the retaining member 42. In some embodiments, the core member 40 is formed with varying rigidity or resiliency in an anterior-posterior or latero-lateral direction.

For example, the material properties of the cured biomaterials 56a, 56b can be selected to determine the amount of rigidity or a resiliency of the core member 40. In some embodiments, an anterior portion 118a of the core member 40 corresponding to the first compartment 50a is formed with a more or less rigid biomaterial than a posterior portion 118b of the core member 40 that corresponds to the second compartment 50b, such that the anterior and posterior portions 118a, 118b of the core member 40 have varying rigidity/resilience to deformation. The core member 40 can similarly be adapted to vary in rigidity/resiliency in the latero-lateral direction as well. In some embodiments, the rigidities are selected to help conform the intervertebral space 22 to a desired amount of lordotic or kyphotic curvature between the first and second vertebrae 24, 26, for example by limiting or controlling an amount of anterior or posterior deflection of the core member 40.

As alluded to above, the core member 40 is adapted to support spinal loads. In some embodiments, the retaining member 42 is also characterized as load bearing and supports a portion of the spinal loads. For example, where the retaining member 42 also incorporates a spring action, the core member 40 and the retaining member 42 each share a portion of the spinal loading. In other embodiments, the retaining member 42 is characterized as non-load bearing and transfers most or all of the spinal loads to the core member 42. The retaining member 42 is non-load bearing, for example, where the retaining member 42 does not incorporate a substantial spring action between the upper and lower portions 78, 80.

The retaining member 42 helps prevent migration or expulsion of core member 40 from the intervertebral space 22 under spinal loading conditions. Embodiments including this feature can be particularly useful in applications addressing the cervical vertebrae. In particular, posterior migration or expulsion of prosthetics is often a problem due to the spinal curvature in the cervical region and the loads encountered in the cervical discs, although migration or expulsion in any of the spinal regions is addressable according to embodiments of the invention.

In some embodiments, the retaining member 42 is implanted with the bend 81 oriented posteriorly. The bend 81 interferes with migration or expulsion of the core member 40 in the posterior direction, reducing the risk of paralysis from spinal cord injury or other serious injury. The concave shape(s) 78a, 78b, 80a, 80b(FIGS. 3 and 4) of the retaining member 40 also help prevent anterior and lateral migration or expulsion of the core member 40 from the intervertebral space 22. Furthermore, the prosthetic 30 acts to limit or control lordotic and/or kyphotic curvature reducing the amount of anterior or posterior “squeezing” on the core member 40 that can cause migration or expulsion of the core member 40.

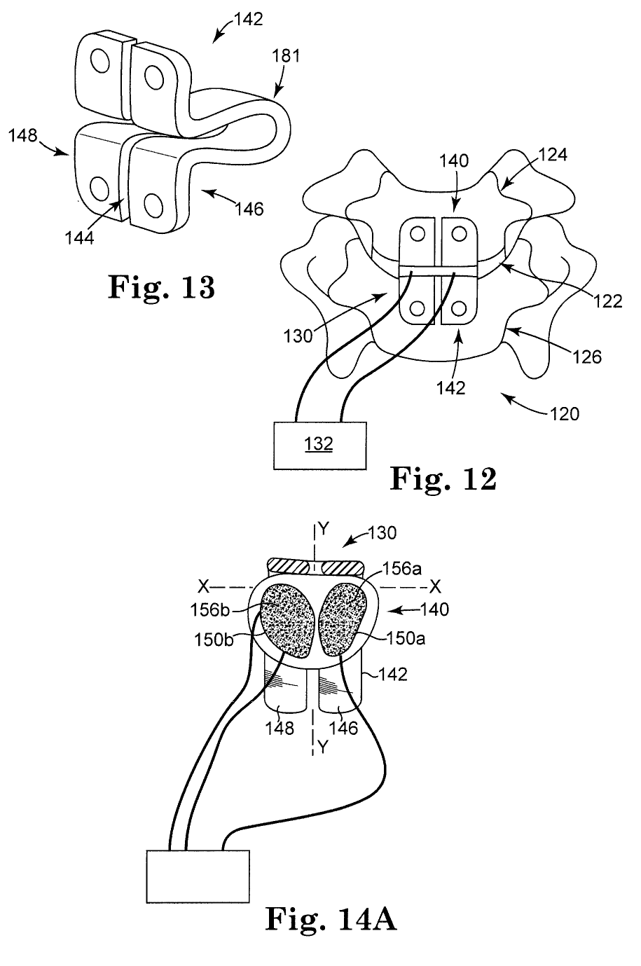

Various embodiments have been described that help facilitate a desired angular offset relative to pitch around the X-axis (FIG. 1) of the intervertebral space 22 (FIG. 1), which otherwise correspond to a desired degree of lordotic or kyphotic curvature between the first and second vertebrae 24, 26 (FIG. 1). FIG. 12 shows another embodiment system 120 for in situ spinal prosthetic formation within an intervertebral space 122 defined between a first vertebra 124 and a second vertebra 126 adjacent the first vertebra 124. In particular, the system 120 is usable to adjust the spacing between the adjacent vertebrae 124, 126 around the Y axis (roll).

The system 120 includes a prosthetic 130 and a biomaterial delivery apparatus 132. The prosthetic 130 includes a core member 140 and a retaining member 142. The biomaterial delivery apparatus 132 and the core member 140 are optionally similar to embodiments of the biomaterial delivery apparatus 32 and the core member 40 previously described. For example, the core member 140 includes first and second compartments 150a, 150b (FIG. 14) for receiving first and second biomaterials 156a, 156b (FIG. 14) according to various embodiments.

FIG. 13 shows the retaining member 142 in greater detail. The retaining member 142 is optionally similar to embodiments of the retaining member 42. The retaining member 142 is shown including a central, longitudinal channel 144 dividing the retaining member 142 into a first lateral portion 146 and a second lateral portion 148. The first and second lateral portions 146, 148 are optionally connected, for example at a bend 181 of the retaining member 142 or are discrete, separate parts as desired. As will be described in greater detail, the longitudinal channel 144 allows the first and second lateral portions 146, 148 to be distracted, or expanded to a different extent, which facilitates adjustment of lateral curvature or roll between the first and second vertebrae 124, 126 (FIG. 12). In some embodiments, adjustment of the lateral curvature or roll is implemented to help correct such disorders as scoliosis, or to fill gaps that created upon resection of tumors, removal of existing implants, or correction of compression fractures, for example.

FIG. 14A is a cross-sectional, top view of the core member 140 and the retaining member 142. The core member 140 is positioned in the retaining member 142 with the first and second compartments 150a, 150b oriented laterally. The core member 140 optionally extends beyond the edges of the retaining member 142, such as illustrated in FIG. 14A. For some applications the retaining member 142 can have a width substantially smaller than a width of the core member 140. The prosthetic 130 is optionally implanted in a similar manner to the prosthetic 30. The first and second biomaterials 156a, 156b are then injected into the first and second compartments 150a, 150b as desired in order to adjust the lateral curvature or roll around the Y axis of the first and second vertebrae 124, 126 (FIG. 12). In particular, the amount of the first and second biomaterials 156a, 156b control a relative amount of distraction of the first and second lateral portions 146, 148, respectively, of the retaining member 142 which is translated to the first and second vertebrae 124, 126.

Lateral adjustment of the prosthetic 130 is useful in a variety of scenarios, such as where a patient is suffering from an abnormal lateral curvature of the spine or where portions of one or both of the first and second vertebrae 124, 126 have been removed, weakened, or otherwise require greater spacing or reinforcement on one lateral side of the intervertebral space 122 (FIG. 12).

FIG. 14B is cross-sectional, top view of an alternate core member 140′ and the retaining member 142′. The core member 140′ includes first, second and third compartments 150a′, 150b′, 150c′ (collectively 150′). Compartments 150a′ and 150b′ are positioned adjacent the first and second portion 146′ and 148′. Compartment 150c′ is located adjacent to the bend 181′. Biomaterials 156a′, 156b′, 156c′ (collectively 156′) are injected into the first, second and third compartments 150a, 150b′, 150c′, respectively. The biomaterial delivery apparatus 132 controls the pressure and/or volume of biomaterial 156 in each compartment 150, so the surgeon can adjust pitch and roll of the adjacent vertebrae 124, 126. The relative pressure and/or volume of biomaterials 156a′ and 156b′ can be used to adjust the lateral curvature or roll around the Y axis of the first and second vertebrae 124, 126 (FIG. 12). The relative pressure and/or volume of biomaterials 156a′, 156b′ vs. 156c′ can be used to adjust the pitch around the X axis of the first and second vertebrae 124, 126 (FIG. 12). Biomaterials 156a′, 156b′, 156c′ can be the same or different materials.

FIG. 14C is cross-sectional, top view of an alternate core member 140″ and the retaining member 142″. The core member 140″ includes first, second, third and fourth compartments 150a″, 150b″, 150c″, 150d″ (collectively 150″). Compartments 150a″ and 150b″ are positioned adjacent the first and second portion 146″ and 148″. Compartments 150c″ and 150d″ are located adjacent to the bend 181′. Biomaterials 156a″, 156b″, 156c″, 156d″ (collectively 156″) are injected into the first, second, third and fourth compartments 150a″, 150b″, 150c″, 150d″ respectively. The biomaterial delivery apparatus 132 controls the pressure and/or volume of biomaterial 156 in each compartment 150, so the surgeon can adjust pitch, roll and yaw of the adjacent vertebrae 124, 126. The relative pressure and/or volume of biomaterials 156a″ and 156b″ can be used to adjust the lateral curvature or roll around the Y axis of the first and second vertebrae 124, 126 (FIG. 12). The relative pressure and/or volume of biomaterials 156a″, 156b″ vs. 156c″ and 156d″ can be used to adjust the pitch around the X axis of the first and second vertebrae 124, 126 (FIG. 12). Biomaterials 156a″, 156b″, 156c″ 156d″ can be the same or different materials.

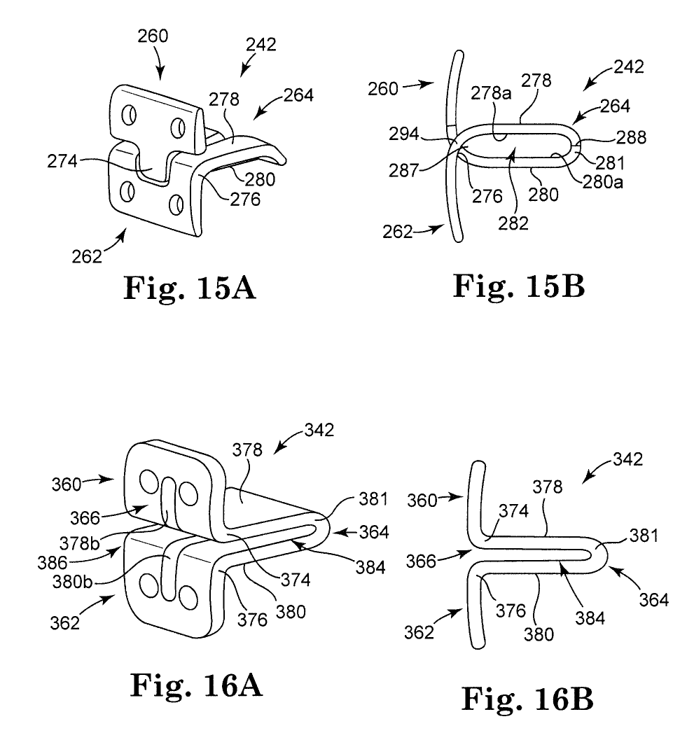

FIGS. 15A-18B are perspective and side views of other retaining members usable in association with embodiments of the invention. FIGS. 15A and 15B show another retaining member 242 from perspective and side views, respectively. With combined reference to FIGS. 15A and 15B, the retaining member 242 is shown including a first flange 260, a second flange 262, and an intermediate body 264 extending between the first and second flanges 260, 262.

The first and second flanges 260, 262 are shown positioned opposite one another, each extending fluidly from the intermediate body 264 in opposite directions from one another. Each of the first and second flanges 260, 262 is T-shaped in front profile and is substantially arcuate in side profile or otherwise adapted to fit against, or track the profile of a vertebra (not shown). For example, the first and second flanges 260, 262 are substantially convex in an anterior direction when viewed from the side, such that each of the flanges 260, 262 has a shape that substantially conforms to the anterior faces of first and second vertebrae (not shown).

As shown, the intermediate body 264 has a recurved shape, the intermediate body extending through an arcuate path back onto itself. In particular, the intermediate body 264 extends from a first end 274 to a second end 276 and includes intersecting upper 278 and lower portions 280 that have an overlapping-loop configuration. The first end 274 is fluidly connected to the first flange 260 while the second end 276 is fluidly connected to the second flange 262.

The upper and lower portions 278, 280 each have inwardly concave shapes 278a, 280a from a side profile (FIG. 12B). In some embodiments, the upper and lower portions 278, 280 are shaped to substantially conform or otherwise track with opposing vertebrae endplates. The upper and lower portions 278, 280 are connected at a bend 281 and combine to define an interior 282 adapted to receive a core member (not shown), such as the core member 40. The interior 282 has an open first side 284 and an open second side 286 opposite the first side 284, a closed front 287, and a closed back 288.

In some embodiments, the intermediate body 264 incorporates some flex, or a spring action between the upper and lower portions 278, 280 as described in association with previous embodiments. In other embodiments, the intermediate body 264 does not exhibit a spring action following implantation as described previously in association with other embodiments.

The closed front and back 287, 288 of the interior 282 and/or the spring action help retain an associated core member (not shown) within the interior 282 following implantation. In particular, the spring action of the retaining member 242 can help prevent core member migration or expulsion from an intervertebral space (not shown) by controlling or limiting the relative angle between the vertebrae forming the intervertebral space. Additional or alternate features such as those previously described can also be employed to reduce the possibility of core member migration or expulsion from the interior 282.

FIGS. 16A and 16B show another retaining member 342 from perspective and side views, respectively. With combined reference to FIGS. 16A and 16B, the retaining member 342 is shown including a first flange 360, a second flange 362, and an intermediate body 364 extending between the first and second flanges 360, 362.

The first and second flanges 360, 362 are positioned opposite one another, each extending fluidly from the intermediate body 364 in opposite directions. Each of the first and second flanges 360, 362 is generally U-shaped in front profile and substantially arcuate in side profile or otherwise adapted to fit against, or track the profile of a vertebra (not shown). For example, the first and second flanges 360, 362 are substantially convex in an anterior direction when viewed from the side, such that each of the flanges 360, 362 has a shape that substantially conforms to the anterior faces of first and second vertebrae (not shown). The first and second flanges 360, 362 also combine to form a central, substantially vertical slot 366. The slot 366 is optionally adapted to receive a core member (not shown) such as the core member 40.

The intermediate body 364 has a recurved shape, the intermediate body 364 extending through an arcuate path from a first end 374 to a second end 376. The intermediate body 364 includes an upper portion 378 and a lower portion 380. The first end 374 of the intermediate body 364 is fluidly connected to the first flange 360 while the second end 376 is fluidly connected to the second flange 362.

The upper and lower portions 378, 380 are each cup-shaped, having inwardly concave shapes 378a, 380a from a side profile (FIG. 16B) and inwardly concave shapes 378b, 380b in front cross-section (shown partially obscured in the perspective view of FIG. 16B). The concave shapes 378b, 380b are substantially continuous with the vertical slot 366 formed by the first and second flanges 360, 362. In some embodiments, the upper and lower portions 378, 380 are shaped to substantially conform or otherwise track with a concavity of the endplates of adjacent vertebrae (not shown).

The upper and lower portions 378, 380 are connected at a bend 381 and combine to define an interior 382 adapted to receive an associated core member (not shown). The interior 382 has an open first side 384, an open second side 386 opposite the first side 384, a front corresponding to the slot 366, and a closed back 388.

In some embodiments, the intermediate body 364 incorporates some flex, or a spring action between the upper and lower portions 378, 380 similarly to previously described embodiments. In other embodiments, the intermediate body 364 does not exhibit a spring action following implantation similarly to other previously described embodiments.

The closed back 388, the concave shapes 378a, 378b, 380a, 380b of the upper and lower portions 378, 380, and/or the spring action help retain an associated core member (not shown) within the interior 382 following implantation. In some embodiments, the retaining member 342 is particularly suited to receiving a core member oriented vertically and received through the slot 366 into the interior 382. Additional or alternate features such as those previously described can also be employed to reduce the possibility of core member migration or expulsion from the interior 382, thus reducing the possibility of core member migration or expulsion from the intervertebral space 22.

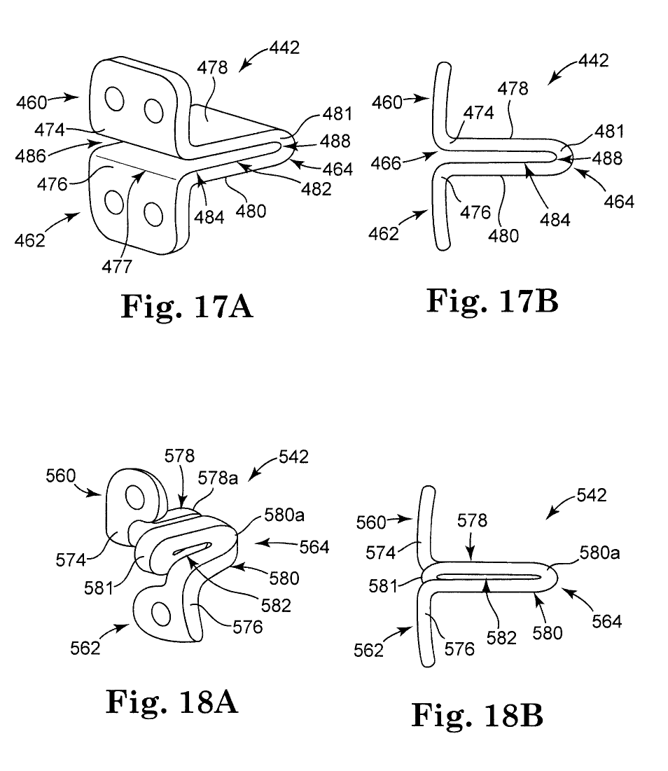

FIGS. 17A and 17B show a retaining member 442 from perspective and side views, respectively. With combined reference to FIGS. 17A and 17B, the retaining member 442 includes a first flange 460, a second flange 462, and an intermediate body 464 extending between the first and second flanges 460, 462.

The first and second flanges 460, 462 are shown positioned opposite one another, each extending fluidly from the intermediate body 464 in opposite directions. Each of the first and second flanges 460, 462 is generally U-shaped in front profile and is substantially arcuate in side profile or otherwise adapted to fit against, or track the outer profile of opposing vertebrae (not shown). For example, the first and second flanges 460, 462 are substantially convex in an anterior direction when viewed from the side, such that each of the flanges 460, 462 has a shape that substantially conforms to the anterior faces of first and second vertebrae (not shown).

The intermediate body 464 has a recurved shape, a portion of the intermediate body 464 extending through an arcuate path from a first end 474 to a second end 476. The intermediate body 464 includes an upper portion 478 and a lower portion 480. The first end 474 is fluidly connected to the first flange 460 while the second end 476 is fluidly connected to the second flange 462 with a gap 477 defined between the first and second ends 474, 476.

The upper and lower portions 478, 480 are each substantially planar from a side profile (FIG. 17B) and in front cross-section (shown partially obscured in the perspective view of FIG. 17B). The upper and lower portions 478, 480 are arcuately connected at a bend 481 and combine to define an interior 482 adapted to receive an associated core member (not shown). The interior 482 has an open first side 484, an open second side 486 opposite the first side 484, an open front corresponding to the gap 466, and a closed back 488.

In some embodiments, the intermediate body 464 incorporates some flex, or a spring action between the upper and lower portions 478, 480 as described in association with previous embodiments. In other embodiments, the intermediate body 464 does not exhibit a spring action following implantation as described previously in association with other embodiments.

The closed back 488 and/or spring action helps retain an associated core member (not shown) within the interior 482 as previously described. Additional or alternate features such as those previously described can also be employed to reduce the possibility of migration or expulsion of a core member from the interior 482.

FIGS. 18A and 18B show a retaining member 542 from perspective and side views, respectively. With combined reference to FIGS. 18A and 18B, the retaining member 542 includes a first flange 560, a second flange 562, and an intermediate body 564 extending between the first and second flanges 560, 562.

The first and second flanges 560, 562 are shown positioned opposite one another, each extending fluidly from the intermediate body 564 in opposite directions. Each of the first and second flanges 560, 562 is substantially arcuate in side profile or otherwise adapted to fit against, or track the profile of a vertebra (not shown). For example, the first and second flanges 560, 562 are substantially convex in an anterior direction when viewed from the side, such that each of the flanges 560, 562 has a shape that substantially conforms to the anterior faces of first and second vertebrae (not shown).

The intermediate body 564 extends from a first end 574 to a second end 576, the first end 574 being fluidly connected to the first flange 560 and the second end 576 being fluidly connected to the second flange 562. The intermediate body 564 has a recurved shape. In one embodiment, the intermediate body 564 extends through an arcuate path back onto itself through two 360 degree turns.

The upper and lower portions 578, 580 each have a recurved shape, defining bends 578a, 580a from a side profile (FIG. 18B). In some embodiments, the upper and lower portions 578, 580 are shaped to substantially conform or otherwise track with a vertebra (not shown). The upper and lower portions 578, 580 are connected at a bend 581 oriented between the first and second flanges 560, 562. The upper and lower portions 578, 580 combine to define an interior 582 adapted to receive a core member, such as one similar to the core member 40. The interior 582 has an open first side 584, an open second side 586 opposite the first side 584, a closed front 587, and a closed back 588.

In some embodiments, the intermediate body 564 incorporates some flex, or a spring action between the upper and lower portions 578, 580 as described in association with previous embodiments. In a related embodiment, the dual-recurved shape of the intermediate body 564, including the bends 581, 578a, and 578b, facilitates greater range of flexing, at both posterior and anterior locations. In other embodiments, the intermediate body 564 does not exhibit a spring action following implantation similarly to embodiments previously described.

The closed front and back 587, 588 of the interior 582 and/or the spring action of the retaining member 542 help retain a core member (not shown) within the interior 582 following implantation. Additional or alternate features such as those previously described can also be employed to reduce the possibility of migration or expulsion of the core member from the interior 582.

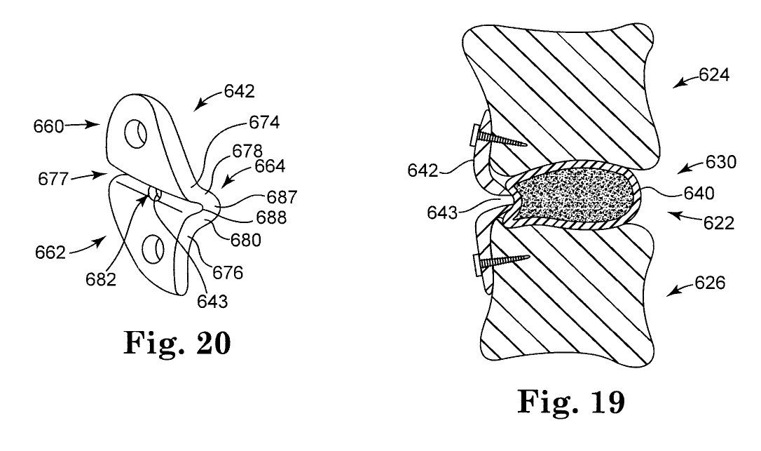

FIG. 19 is a perspective view of another spinal prosthetic 630 including a core member 640 and a retaining member 642 implanted and formed within an intervertebral space 622 defined between a first vertebra 624 and second vertebra 626. The core member 640 is similar to embodiments of the core member 40 previously described. FIG. 20 shows the retaining member 642 from a perspective view. With combined reference to FIGS. 19 and 20, the retaining member 642 is shown including a first flange 660, a second flange 662, and an intermediate body 664 extending between the first and second flanges 660, 662.

The first and second flanges 660, 662 are shown positioned opposite one another, each extending fluidly from the intermediate body 664 in opposite directions to one another. Each of the first and second flanges 660, 662 is generally U-shaped in front profile and is substantially arcuate in side profile or otherwise adapted to fit against, or track the profile of the first and second vertebrae 624, 626 respectively. For example, the first and second flanges 660, 662 are substantially convex in an anterior direction when viewed from the side, such that each of the flanges 660, 662 has a shape that substantially conforms to the anterior faces of first and second vertebrae 624, 626.

As shown, the intermediate body 664 has a recurved shape and extends through an arcuate path from a first end 674 to a second end 676. The intermediate body 664 is adapted to be at least partially disposed in the intervertebral space 622 and includes an upper portion 678 and a lower portion 680. The first end 674 is fluidly connected to the first flange 660 while the second end 676 is fluidly connected to the second flange 662, a gap 677 being defined between the first and second ends 674, 676.

The upper and lower portions 678, 680 are arcuately connected at a bend 681 and combine to define an interior or recess 682. The interior 682 has an open first side 684, an open second side 686 opposite the first side 684, and an open front corresponding to the gap 677, and a closed back 688.

In some embodiments, the intermediate body 664 incorporates some flex, or a spring action between the upper and lower portions 678, 680 as described in association with previous embodiments. In other embodiments, the intermediate body 664 does not exhibit a spring action following implantation similarly to embodiments previously described.

As shown in FIG. 19, the core member 640 is abutted against and/or secured to the bend 681 of the retaining member 642, which, in turn, is secured to the first and second vertebrae 624, 626 using bone screw or other fasteners, for example. The retaining member 642 interferes with anterior migration or expulsion of the core member 640, as the core member 640 is disposed behind the retaining member 642, within the intervertebral space 622. In particular, the retaining member 642 blocks movement of the core member 640 in the direction of implantation. In one embodiment, the retaining member 642 includes a hole 643 through which a mold for the core member 640 can be inserted.

The spring action of the retaining member 642 optionally helps prevent the core member 640 from expelling or migrating in a posterior direction from the intervertebral space 622 by controlling or limiting the relative angle between the vertebrae 624, 626 in a similar manner to embodiments previously described. In particular, the spring action of the retaining member 642 helps limit the lordotic curvature of the first and second vertebra 624, 626 which would otherwise promote posterior migration or expulsion of the core member 640 from the intervertebral space 622. In other embodiments, the spring-action of the retaining member 642 helps limit the kyphotic curvature of the first and second vertebra 624, 626.

As previously referenced, the core member 640 is also optionally secured to the retaining member 642, for example via adhesives, sutures, clips, or other fasteners to help prevent posterior migration or expulsion of the core member 640 from the intervertebral space 622. Additional or alternate features such as those previously described can also be employed to reduce the possibility of migration or expulsion of the core member 640 from the intervertebral space 622.

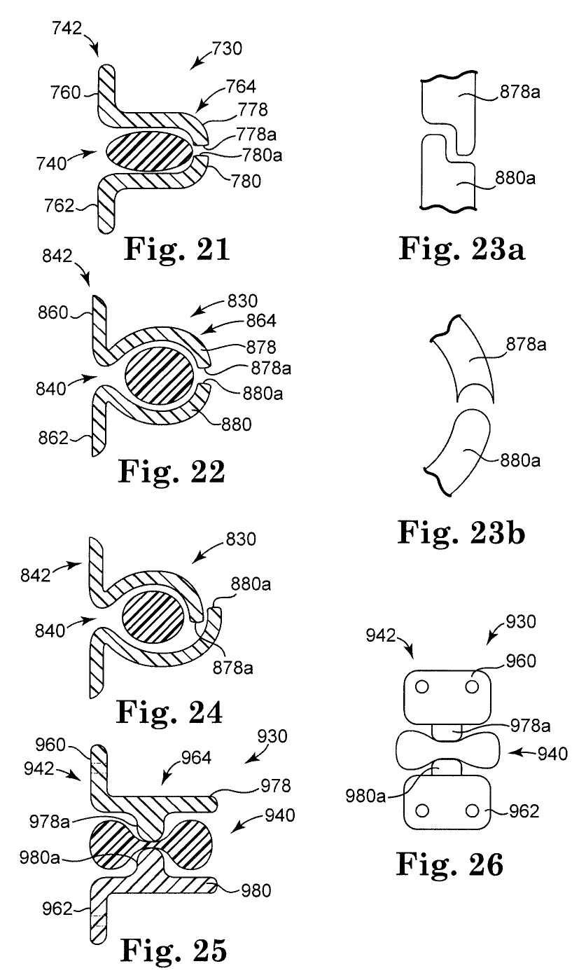

FIG. 21 is a sectional side view of another spinal prosthetic 730 including a core member 740 and a retaining member 742 for supporting an intervertebral space, such as that shown in FIG. 1. The core member 740 is similar to embodiments of the core member 40 previously described. The retaining member 742 includes a first flange 760, a second flange 762, and an intermediate body 764 formed of an upper portion 778 and a lower portion 780 extending from the first and second flanges 760, 762, respectively. The flanges 760, 762 are secured to vertebrae forming the intervertebral space via a variety of means, including screws, adhesives, tissue ingrowth and others. As shown, the upper and lower portions 778, 780 terminate at ends 767a, 776b which either define a gap or contact in a manner that allows relative vertical movement of the portions 778, 780. As shown, the upper and lower portions 778, 780 are adapted to help reduce the risk of migration or expulsion of the core member 740 from the retaining member 742 and thus, the intervertebral space.

FIG. 22 is a sectional side view of another spinal prosthetic 830 substantially similar to the spinal prosthetic 730. The spinal prosthetic 830 includes a core member 840 and a retaining member 842 for supporting an intervertebral space, such as that shown in FIG. 1. The core member 840 is similar to embodiments of the core member 40 previously described. The retaining member 842 includes a first flange 860, a second flange 862, and an intermediate body 864 formed of an upper portion 878a and a lower portion 880a extending from the first and second flanges 860, 862, respectively. When compressed, the upper portion 878a and a lower portion 880a may slide past each other. The flanges 860, 862 are secured to vertebrae forming the intervertebral space via a variety of means, including screws, adhesives, tissue ingrowth and others.

As shown, the upper and lower portions 878, 880 cup inwardly toward one another opposite the flanges 860, 862. When compressed, the upper and lower portions 878, 880 may engage to limit further displacement. The upper and lower portions 878, 880 terminate at ends 867a, 876b which define a gap or contact in a manner that allows relative vertical movement of the portions 878, 880 along the axis of the spine. As shown, the upper and lower portions 878, 880 are adapted to help reduce the risk of migration or expulsion of the core member 840 from the retaining member 842 and thus, the intervertebral space. FIGS. 23a and 23b show two exemplary configurations for the ends 867a, 867b of the retaining member 842. FIG. 23a shows an overlapping tooth configuration, while FIG. 23b shows a cup and ball configuration. A variety of configurations of the ends 867a, 867b are contemplated and are applicable to other embodiments described herein. FIG. 24 shows another configuration of the spinal prosthetic 830 where the ends 876a, 867b are adapted to overlap.

FIG. 25 is a sectional side view of another spinal prosthetic 930 and FIG. 26 is a front view of the prosthetic 930. As shown in FIGS. 25 and 26, the spinal prosthetic 930 includes a core member 940 and a retaining member 942 for supporting an intervertebral space, such as that shown in FIG. 1. The core member 940 is similar to embodiments of the core member 40 previously described. The retaining member 942 includes a first flange 960, a second flange 962, and an intermediate body 964 formed of an upper portion 978 and a lower portion 980 extending from the first and second flanges 960, 962, respectively. The flanges 960, 962 are secured to vertebrae forming the intervertebral space via a variety of means, including screws, adhesives, tissue ingrowth and others. As shown, the upper and lower portions 978, 980 each include central projections 969a, 969b extending toward one another with a central portion of core member 940 engaged or otherwise retained by the central projections 969a, 969b. As shown, the core member 940 optionally includes a bi-concave center to receive the projections according to some embodiments. In other embodiments, the core member 940 has a central lumen (not shown), or is “doughnut shaped” to receive one or both of the projections 969a, 969b. Regardless, the upper and lower portions 978, 980 are adapted to help reduce the risk of migration or expulsion of the core member 940 from the retaining member 942 and thus, the intervertebral space.

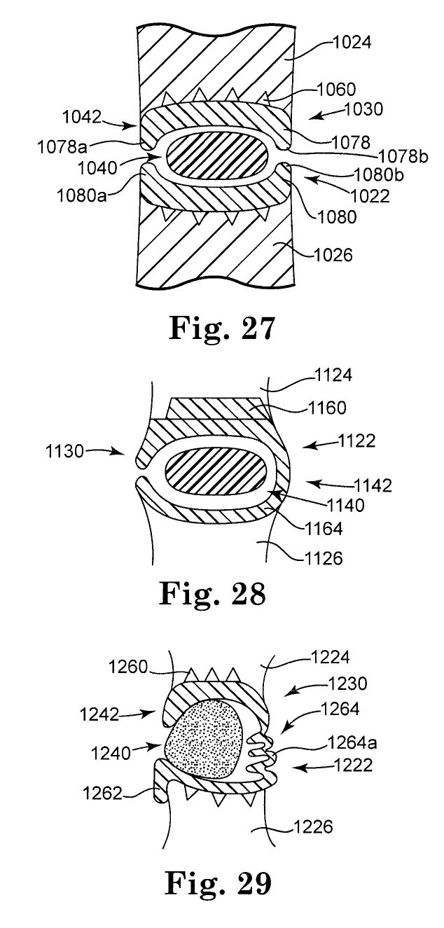

FIG. 27 shows another spinal prosthetic 1030 including a core member 1040 and a retaining member 1042 for supporting an intervertebral space 1022 defined between a first vertebra 1024 and second vertebra 1026. The core member 1040 is similar to embodiments of the core member 40 previously described. Although some embodiments include flanges for securing the retaining member to vertebrae, as shown in FIG. 27, some embodiments additionally or alternative include projections 1060, such as spikes, for securing the retaining member 1042 in the intervertebral space 1022. The retaining member 1042 is shown including an upper portion 1078 substantially concave down in shape and a lower portion 1080 substantially concave up in shape. The upper and lower portions 1078, 1080 are preferably discrete pieces. Protrusions 1078a, 1078b, 1080a, 1080b are adapted to help reduce the risk of migration or expulsion of the core member 1040 from the retaining member 1042 and thus, the intervertebral space 1022.

FIG. 28 shows another spinal prosthetic 1130 including a core member 1140 and a retaining member 1142 for supporting an intervertebral space 1122 defined between a first vertebra 1124 and second vertebra 1126. The core member 1140 is similar to embodiments of the core member 40 previously described. As shown in FIG. 28, the retaining member 1142 includes a keel 1160 formed as a substantially elongate, rectangular projection, for securing the retaining member 1142 in the intervertebral space 1122, for example to the endplate of the upper vertebra 1124. If desired, a second keel (not shown) is incorporated for securing the retaining member 1142 to the lower vertebra 1126. As shown, the retaining member 1142 includes a substantially C-shaped intermediate body 1164 adapted to help reduce the risk of migration or expulsion of the core member 1140 from the retaining member 1142 and thus, the intervertebral space 1122.

FIG. 29 shows another spinal prosthetic 1230 including a core member 1240 and a retaining member 1242 for supporting an intervertebral space 1222 defined between a first vertebra 1224 and second vertebra 1226. The core member 1240 is similar to embodiments of the core member 40 previously described. As shown in FIG. 29, the retaining member 1242 includes projections 1260, such as spikes, for securing the retaining member 1242 in the intervertebral space 1222, for example to the endplates of the vertebrae 1224, 1226. The retaining member 1242 also includes at least one flange 1262 for securing the retaining member 1242 to the vertebra 1226, for example using fastener(s) as previously described. The retaining member 1242 also includes a substantially C-shaped intermediate body 1264 with a hinge portion 1264a that is substantially flexible in nature, for example being formed of an elastic or compliant material and/or having the accordion-shape shown in FIG. 29. As with other embodiments, the intermediate body 1264, including the hinge portion 1264a, is adapted to help reduce the risk of migration or expulsion of the core member 1240 from the retaining member 1242 and thus, the intervertebral space 1222.

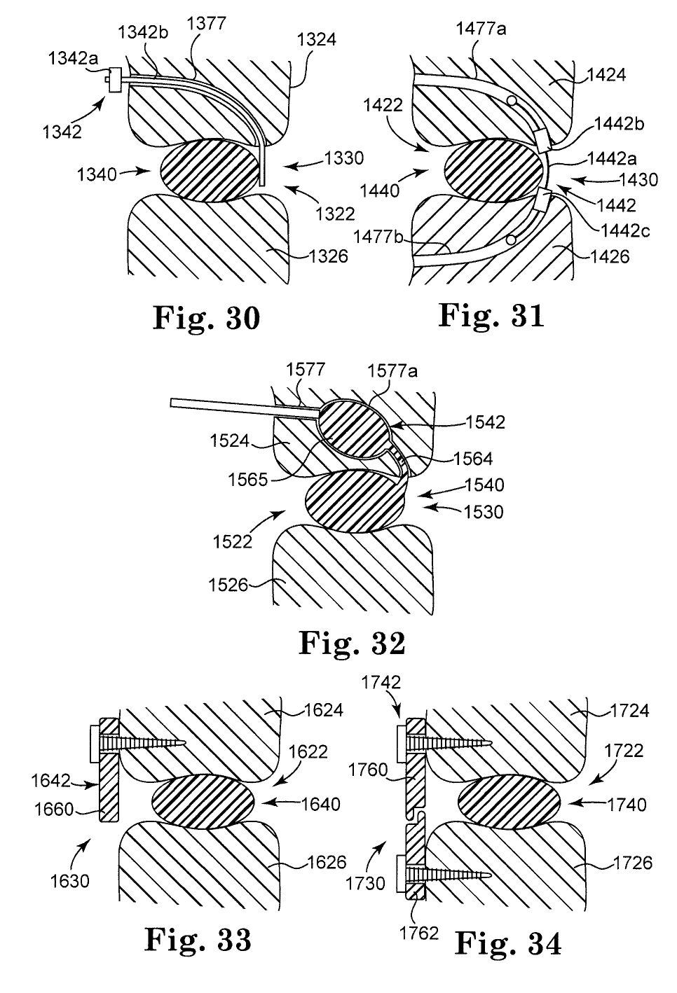

FIG. 30 shows another spinal prosthetic 1330 including a core member 1340 and a retaining member 1342 for supporting an intervertebral space 1322 defined between a first vertebra 1324 and second vertebra 1326. The core member 1340 is similar to embodiments of the core member 40 previously described. As shown, the retaining member 1342 is formed as an elongate rod or plate 1342b and includes an end retainer 1342a, such as an endcap or nut. The retaining member 1342 is inserted through a channel 1377 formed into one of the vertebrae, the first vertebra 1324 in FIG. 29, and extends into the intervertebral space 1322. The end retainer 1342a acts to secure the retaining member 1342 to the vertebra 1326, for example to the outer face of the vertebra 1326. As shown, the retaining member 1342 is substantially arcuately-shaped, extending into the intervertebral space 1322 to help reduce the risk of migration or expulsion of the core member 1340 from the intervertebral space 1322 by blocking migration of the core member 1340.

FIG. 31 shows another spinal prosthetic 1430 including a core member 1440 and a retaining member 1442 for supporting an intervertebral space 1422 defined between a first vertebra 1424 and second vertebra 1426. The core member 1440 is similar to embodiments of the core member 40 previously described. The retaining member 1442 includes an elongate member 1442a, which can be rod-like or plate-like in form, for example. The retaining member 1442 also includes a first end retainer 1442b and a second end retainer 1442c which are both adapted to slidably receive the elongate member 1442a, for example being formed as hollow tubules or other appropriately shaped receptacles. As shown, the elongate member 1442a optionally includes thickened ends to keep the elongate member 1442a secured within the end retainers 1442b, 1442c.

The two end retainers 1442b, 1442c are secured in channels 1477a, 1477b formed into the first and second vertebrae 1424, 1426, respectively, using adhesives or other fastening means, for example. The elongate member 1442a is then received through the two end retainers 1442b, 1442c, so that the elongate member 1442a will not overly restrict relative movement (e.g., pitch and/or yaw) between the vertebrae 1424, 1426 while still being adapted to help reduce the risk of migration or expulsion of the core member 1440 from the intervertebral space 1422.

FIG. 32 shows another spinal prosthetic 1530 including a core member 1540 and a retaining member 1542 for supporting an intervertebral space 1522 defined between a first vertebra 1524 and second vertebra 1526. The core member 1540 is similar to embodiments of the core member 40 previously described. The retaining member 1542 includes an intermediate body 1564, which is optionally catheter-like in form. The retaining member 1542 also includes a main body 1565 which is optionally substantially similar to the core member 1540.

In use, and as shown, a channel 1577 is formed through the first vertebra 1524 (though the second vertebra 1526 is also an option) to the intervertebral space 1522, where the channel 1577 includes a hollowed out portion 1577a. The core member 1540 is directed through the channel 1577 to the intervertebral space 1522 and the retaining member 1542 is positioned in the hollowed out portion 1577a as shown. A biomaterial delivery apparatus (see, e.g., FIG. 6), or other appropriate device is used to inflate the retaining member 1542, as well as the core member 1540. In particular, biomaterial or other appropriate material is injected into the main body 1565, through the intermediate body 1564, and into the core member 1540. Upon inflation, the retaining member 1542 helps reduce the risk of migration or expulsion of the core member 1540 from the intervertebral space 1522 as the core member 1540 is tied to the retaining member via the intermediate body 1564.

FIG. 33 shows another spinal prosthetic 1630 including a core member 1640 and a retaining member 1642 for supporting an intervertebral space 1622 defined between a first vertebra 1624 and second vertebra 1626. The core member 1640 is similar to embodiments of the core member 40 previously described. The retaining member 1642 includes an elongate flange 1660 extending adjacent the intervertebral space 1622 to help reduce the risk of migration or expulsion of the core member 1640 from the intervertebral space 1622.

FIG. 34 shows another spinal prosthetic 1730 including first and second elongate flanges 1760, 1762, respectively extending adjacent the intervertebral space 1722 to help reduce the risk of migration or expulsion of core member 1740 from the intervertebral space 1722. The flanges 1760, 1762 are adapted to contact in a manner that allows relative vertical movement of the flanges 1760, 1762. The adjacent ends of the flanges 1760, 1762 optionally interact in an overlapping tooth configuration as shown, in a cup and ball configuration, or any of a variety of other configurations, for example, the flanges 1760, 1762 are alternatively adapted to overlap as noted in association with other embodiments described herein.

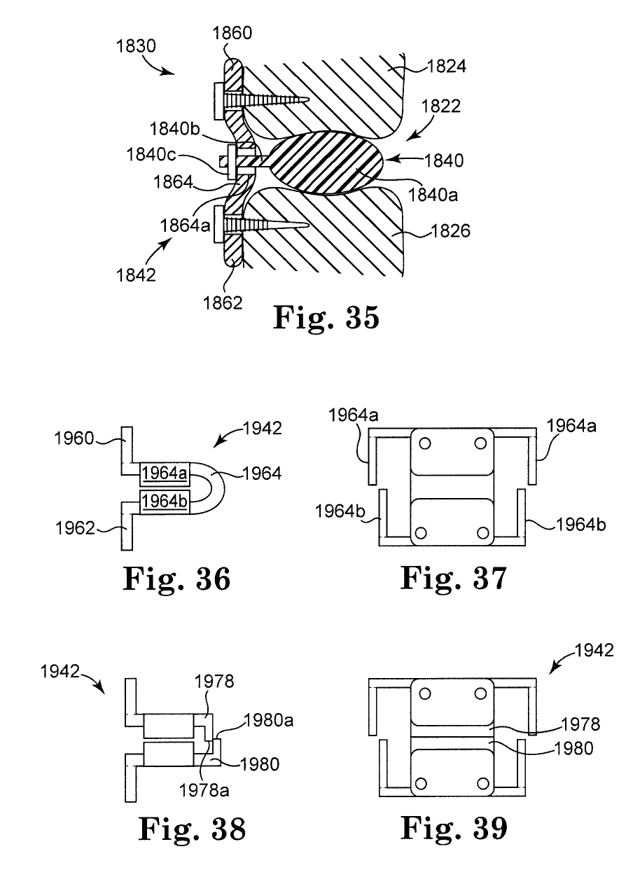

FIG. 35 shows another spinal prosthetic 1830 including a core member 1840 and a retaining member 1842 for supporting an intervertebral space 1822. The core member 1840 is similar to embodiments of the core member 40 previously described, and includes a main body 1840a and a tail 1840b extending from the main body 1840a. The retaining member 1842 includes a first flange 1860, a second flange 1862, and an intermediate body 1864 with a hole 1864a adapted to receive the tail 1840b. The flanges 1860, 1862 are secured to vertebrae forming the intervertebral space via a variety of means, including screws, adhesives, tissue ingrowth and others. As shown, the tail 1840b is secured through the hole 1864a in the flange 1864 with a clip or nut 1840c. The intermediate body 1864 is adapted to help reduce the risk of migration or expulsion of the core member 1840 from the intervertebral space 1822, with the intermediate body 1864 acting as a physical barrier to migration toward the intermediate body 1864 and the tail 1840b acting as a tether to the intermediate body 1864 helping prevent migration away from the intermediate body 1864.

FIGS. 36 and 37 show a retaining member 1942 of another spinal prosthetic where FIG. 36 is a sectional side view and FIG. 37 is a front view of the retaining member 1942. The retaining member 1942 includes a first flange 1960, a second flange 1962, and an intermediate body 1964 having a first pair of side walls 1964a and a second pair of side walls 1964b extending upwardly toward the first pair of sidewalls 1964a. The first and second pairs of sidewalls 1964a, 1964b are generally adapted to help prevent migration or expulsion of a core member (not shown) from one or both sides of the retaining member 1942.

As shown in FIG. 37, the first pair of sidewalls 1964a are optionally laterally offset from the second pair of sidewalls 1964b such that the first and second flanges 1960, 1962 can be compressed toward one another without the sidewalls 1964a, 1964b interfering. In other embodiments, the sidewalls 1964a, 1964b are substantially aligned to limit the maximum amount of compression that the retaining member 1942 undergoes. It should be noted that the retaining member 1942, along with various other embodiments, is optionally used in a lateral implantation approach, with the sidewalls oriented in the posterior-anterior direction following implantation, although posterior or anterior implantation approaches are also contemplated. Furthermore, as shown in FIGS. 38 and 39, the retaining member is optionally formed of separate, upper and lower portions 1978, 1980 having overlapping ends 1978a, 1980a.

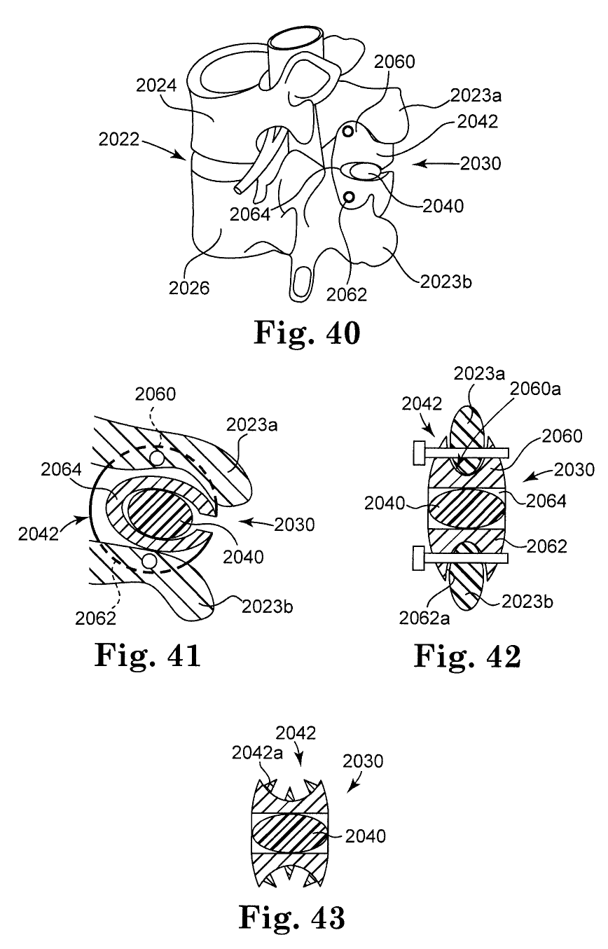

Although the embodiments above have been described with reference to implantation within intervertebral spaces, spinal prosthetics of the present invention are additionally or alternatively implanted outside of intervertebral spaces, for example adjacent the spinous process. FIG. 40 is a perspective view of another spinal prosthetic 2030 including an inflatable core member 2040 and a retaining member 2042 for supporting adjacent first and second spinous processes 2023a, 2023b and indirectly supporting an intervertebral space 2022 via the spinous processes 2023a, 2023b. FIG. 41 is a cross-section, or sectional view, of the prosthetic 2030 taken along a central anterior-posterior plane. FIG. 42 is a cross-section of the prosthetic 2030 taken along a central latero-lateral plane.

With reference to FIGS. 40-42, the inflactable core member 2040 is similar to embodiments of the core member 40 previously described and is received within the retaining member 2042. The retaining member 2042 includes an upper body 2060, a lower body 2062, and an intermediate body 2064 extending fluidly between the upper and lower bodies 2060, 2062. The upper and lower bodies 2060, 2062 define concave surfaces 2060a, 2062a, respectively, for receiving/abutting the first and second spinous processes 2023a, 2023b, respectively. The upper and lower bodies 2060, 2062 are secured to the spinous processes 2023a, 2023b via a variety of means, including screws (as shown), adhesives, tissue ingrowth and others means. The intermediate body 2064 is curved and is optionally substantially spring-like in nature, although flexible, compressible, non-spring-like materials/configurations are contemplated.

Similarly to other embodiments, the retaining member 2042 is optionally implanted with the core member 2040 inserted into the retaining member 2042 and then inflated to a desired shape/size in vivo. As with various other embodiments, the core member 2040 is optionally received in the core member 2040 and/or secured to the retaining member 2042 prior to implantation of the retaining member 2042. During and following inflation, the retaining member 2042 helps reduce the risk of migration or expulsion of the core member 2040 from the retaining member 2042 and thus, from the spinous processes 2023a, 2023b. Although the retaining member 2042 is secured to the spinous processes 2023a, 2023b using fasteners such as screws as best seen in FIG. 42, alternative or additional fastening means are employed as desired. For example, FIG. 43 shows the retaining member 2042 with a plurality of projections 2042a, such as spikes, which additionally or alternatively help secure the retaining member 2042 to the spinous processes 2023a, 2023b.

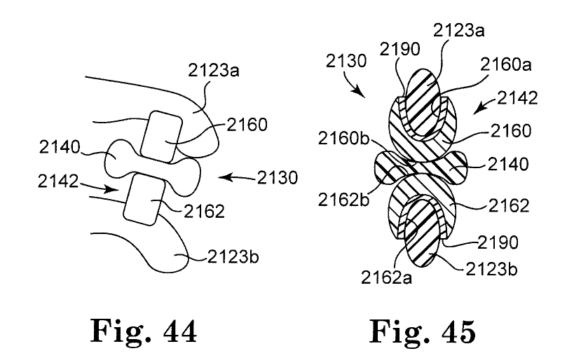

FIG. 44 is a side view of another spinal prosthetic 2130 including a core member 2140 and a retaining member 2142 for supporting adjacent first and second spinous processes 2123a, 2123b and indirectly supporting an intervertebral space via the spinous processes 2123a, 2123b. FIG. 45 is a cross-section of the prosthetic 2130 taken along a central latero-lateral plane. With reference to FIGS. 44 and 45, the spinal prosthetic 2130 includes a core member 2140 and a retaining member 2142. The core member 2140 is similar to embodiments of the core member 40 previously described.

The retaining member 2142 includes an upper body 2160 and a lower body 2162 formed as separate pieces. The upper and lower bodies 2160, 2162 define concave surfaces 2160a, 2162a, respectively, for receiving/abutting the first and second spinous processes 2123a, 2123b, respectively. The upper and lower bodies 2160, 2162 each have terminal ends 2160b, 2162b and are secured to the spinous processes 2123a, 2123b via a variety of means, including adhesives 2190 (as shown), screws, tissue ingrowth and others means. As shown, the terminal ends 2160b, 2162b of the upper and lower bodies 2160, 2162 project toward one another with a central portion of core member 2140 engaged or otherwise retained between the ends 2160b, 2162b. As shown, the core member 2140 optionally includes a bi-concave center to receive the ends 2160b, 2162b according to some embodiments. In other embodiments, the core member 2140 has a central lumen (not shown), or is “doughnut shaped” to receive one or both of the ends 2160b, 2162b. Regardless, the retaining member 2142 is adapted to help reduce the risk of migration or expulsion of the core member 2140 from the retaining member 2142 and thus, from migrating or expelling from between the spinous processes 2123a, 2123b. As described in association with other embodiments, the core member 2140 can also be adhered or otherwise further secured to the retaining member 2142 as desired.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What is claimed is:

1. A system for forming a spinal prosthesis in situ within an intervertebral space located between first and second adjacent vertebrae, the system comprising:
at least one mold comprising at least one internal compartment adapted to receive at least one flowable biomaterial;
a retaining member adapted to secure the mold between the first and second vertebrae, the retaining member comprising;
a first portion adapted to be engaged with a first surface of the first vertebra;
a second portion adapted to be engaged with a second surface of the second vertebra; and
an intermediate body operatively coupling the first portion to the second portion, the intermediate body adapted to be positioned in or adjacent to the intervertebral space;
a biomaterial delivery apparatus in fluid communication with the mold at a pressure sufficient for the mold to engage with the retaining member, wherein the spinal prosthesis selectively position the first vertebrae relative to the second vertebrae.

2. The system of claim 1 wherein the mold comprises at least a first internal compartment and a second internal compartment.

3. The system of claim 1 wherein the mold comprises a first internal compartment located in a posterior portion of an intervertebral disc space and a second internal compartments located in an anterior portion of an intervertebral disc space.

4. The system of claim 1 wherein the mold comprises first internal compartment located on one side of a medio-lateral plane through an intervertebral disc space and a second internal compartments located on the opposite side of the medio-lateral plane.

5. The system of claim 1 wherein the mold comprises a plurality of compartments positioned to adjust at least pitch of the first vertebrae relative to the second vertebrae.

6. The system of claim 1 wherein the mold comprises a plurality of compartments positioned to adjust at least roll of the first vertebrae relative to the second vertebrae.

7. The system of claim 1 wherein the mold comprises a plurality of compartments positioned to adjust at least yaw of the first vertebrae relative to the second vertebrae.

8. The system of claim 1 wherein the retaining member comprises at least two discrete pieces.

9. The system of claim 1 wherein the intermediate body comprises a spring member.

10. The system of claim 1 wherein the intermediate body comprises a flexible member.

11. The system of claim 1 wherein the intermediate body comprises an interior shape adapted to receive the mold in an expanded state.

12. The system of claim 1 wherein the intermediate body comprises a curved shape including a tear-drop shape adapted to receive the mold.

13. The system of claim 1 wherein the intermediate body comprises an upper portion, a lower portion opposite the upper portion, a closed end, an open front end opposite the closed end, an open first side, and an open second side opposite the first side.

14. The system of claim 1 wherein the intermediate body is adapted to prevent at least one of migration and expulsion of the mold from at least one of a posterior direction or an anterior direction.

15. The system of claim 1 wherein the intermediate body is adapted to carry at least a portion of a load imposed by the first vertebrae on the second vertebrae.

16. The system of claim 1 comprising fasteners securing the first and second flanges to the first and second vertebrae, respectively.

17. The system of claim 1 wherein the biomaterial delivery apparatus comprises an endpoint monitor adapted to provide an indication of an endpoint for biomaterial delivery.

18. The system of claim 1 wherein the biomaterial delivery apparatus is adapted to deliver a first biomaterial into a first internal compartment and a second biomaterial into a second internal compartment.

19. The system of claim 1 wherein the biomaterial delivery apparatus comprises a first lumen fluidly coupled to a first internal compartment and a second lumen fluidly coupled to a second internal compartment.

20. The system of claim 1 wherein the system comprises a total disc prosthesis.

21. The system of claim 1 wherein the system comprises one of a cervical disc prosthesis or a lumbar disc prosthesis.

22. The system of claim 1 wherein the system comprises an interspinous process prosthesis.

23. A system for forming a spinal prosthesis in situ within an intervertebral space defined by first and second adjacent vertebrae, each of the first and second vertebrae having an outer surface, the system comprising:
at least one mold comprising at least one internal compartment adapted to receive at least one flowable biomaterial;
a retaining member adapted to secure the mold between the first and second vertebrae, the retaining member comprising;
a first portion adapted to be secured to a first surface of the first vertebra;
a second portion adapted to be secured to a second surface of the second vertebra; and
an engagement region where a portion of the first portion engages the second portion; and
a biomaterial delivery apparatus in fluid communication with the mold at a pressure sufficient for the mold to engage with the retaining member a sufficient amount to selectively position the first vertebrae relative to the second vertebrae.

24. A method of implanting a spinal prosthesis in an intervertebral space defined by a first and a second vertebrae of a patient's spine, the method comprising:
positioning a retaining member in the intervertebral disc space;
engaging a first portion of the retaining member with a surface of the first vertebrae;
engaging a second portion of the retaining member with a surface of the second vertebrae;
positioning a mold including at least one internal compartment adjacent the retaining member and between the first and second vertebrae;
delivering a flowable biomaterial to the mold until the mold engages with the retaining member a sufficient amount to selectively position the first vertebrae relative to the second vertebrae; and
allowing the delivered biomaterial to at least partially cure.

25. The method of claim 24 comprising locating at least two molds in the intervertebral disc space.

26. The method of claim 24 comprising the steps of:
locating a first internal compartment in a posterior portion of the intervertebral disc space; and
locating a second internal compartments located in an anterior portion of the intervertebral disc space.

27. The method of claim 24 comprising the steps of:
locating a first internal compartment on one side of a medio-lateral plane through the intervertebral disc space; and
locating a second internal compartments on the opposite side of the medio-lateral plane.

28. The method of claim 24 comprising the step of controlling the biomaterial pressure and/or volume to adjust at least pitch of the first vertebrae relative to the second vertebrae.

29. The method of claim 24 comprising the step of controlling the biomaterial pressure and/or volume to adjust at least roll of the first vertebrae relative to the second vertebrae.

30. The method of claim 24 comprising the step of controlling the biomaterial pressure and/or volume to adjust at least yaw of the first vertebrae relative to the second vertebrae.

31. The method of claim 24 comprising configuring the retaining member with an interior shape corresponding to the mold in an expanded state.

32. The method of claim 24 comprising locating the retaining member to prevent at least one of migration and expulsion of the mold from at least one of a posterior direction or an anterior direction.

33. The method of claim 24 comprising applying a load from the first and second vertebrae onto the retaining member.

34. The method of claim 24 comprising securing the first and second flanges to the first and second vertebrae, respectively.

35. The method of claim 24 comprising delivering a first biomaterial into a first internal compartment and a second biomaterial into a second internal compartment.

36. The method of claim 24 comprising delivering a first volume of biomaterial to a first internal compartment and a second volume of biomaterial to a second internal compartment.

37. The method of claim 24 comprising delivering biomaterial at a first pressure to a first internal compartment of the mold and biomaterial at a second pressure to a second internal compartment.

38. The method of claim 24 comprising:
loading the intervertebral space with a spinal load; and
supporting a first portion of the spinal load with the retaining member and supporting a second portion of the spinal load with the mold and biomaterial.

39. The method of claim 24 comprising distracting upper and lower portions of the retaining member during delivery of the flowable biomaterial into the mold.

40. The method of claim 24 comprising the step of:
delivering the biomaterial to a first internal compartment in the mold located in a posterior portion of the intervertebral disc space; and
delivering the biomaterial to a second internal compartment in the mold located in an anterior portion of the intervertebral disc space.
